(19) United States
(12) Patent Application Publication
(10) Pub. No.: US 2018/0229055 A1
(43) Pub. Date: Aug. 16, 2018

(54) METHODS AND DEVICES FOR APPLICATION OF BETA RADIATION TREATMENT FOLLOWING GLAUCOMA SURGERY

(71) Applicant: SALUTARIS MEDICAL DEVICES, INC., Tucson, AZ (US)

(72) Inventor: Laurence J. Marsteller, Tucson, AZ (US)

(21) Appl. No.: 15/628,952

(22) Filed: Jun. 21, 2017

Related U.S. Application Data
(63) Continuation of application No. 14/687,784, filed on Apr. 15, 2015, now abandoned.
(60) Provisional application No. 61/980,705, filed on Apr. 17, 2014.

(51) Int. Cl. A61N 5/10

(52) U.S. Cl. CPC A61N 5/1017; A61N 2005/1089; A61N 2005/1094; A61N 2005/1024

(57) ABSTRACT

Methods and devices for the application of beta radiation treatment following trabeculectomy for helping to prevent post-trabeculectomy wound reversion. The devices may feature a handle and a beta radiation source. The beta radiation source may include strontium-90, yttrium-90, potassium-32, or any other appropriate radiation source or combination thereof. The device may be used to expose a trabeculectomy wound to the radiation source following the surgical procedure. In some embodiments, a drug such as 5-fluorouracil or mitomycin c is used in combination.

### CROSS REFERENCE

This application is a continuation and claims benefit of U.S. application Ser. No. 14/687,784 filed Apr. 15, 2015, which is a non-provisional and claims benefit of U.S. Provisional Application No. 61/980,705, filed Apr. 17, 2014, the specification(s) of which is/are incorporated herein in their entirety by reference.

### BACKGROUND OF THE INVENTION

Glaucoma is the world's second most common blinding condition causing irreversible visual loss. Surgery (e.g., trabeculectomy) is often the principal means of managing glaucoma. However, such surgery is often compromised by a healing response (wound reversion). To combat the healing response, anti-metabolites such as 5-fluorouracil (5FU) or mitomycin C commonly prescribed. The present invention features methods and devices for the application of beta radiation treatment following trabeculectomy. Beta radiation may help prevent post-trabeculectomy wound reversion.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY

The present invention features methods and devices for the use of radiation treatment following trabeculectomy. For example, the present invention features a method of preventing post-trabeculectomy wound reversion. In some embodiments, the method comprises providing an applicator, wherein the applicator comprises a handle with a distal end and a beta radiation source disposed on the distal end, and exposing a trabeculectomy wound do the beta radiation source. In some embodiments, the beta radiation source comprises strontium-90, yttrium-90, potassium-32, or a combination thereof. In some embodiments, the beta radiation source helps to prevent wound reversion.

In some embodiments, the applicator further comprises a shield disposed between the radiation source and a proximal end of the handle. In some embodiments, the shield is constructed from a clear, transparent, or translucent material. In some embodiments, the material comprises plastic. In some embodiments, the beta radiation source is sealed. In some embodiments, the beta radiation source comprises a seed. In some embodiments, the beta radiation source provides a radiation dose of about 1000 cGy. In some embodiments, the beta radiation source provides a radiation dose between about 500 to 1200 cGy. In some embodiments, the method further comprises introducing a drug to the trabeculectomy wound after the wound is subjected to the beta radiation source.

The present invention also features an applicator for introducing radiation to a trabeculectomy wound. In some embodiments, the applicator comprises a handle with a distal end and a beta radiation source disposed on the distal end. In some embodiments, the beta radiation source comprises strontium-90, yttrium-90, potassium-32, or a combination thereof.

In some embodiments, the applicator further comprises a shield disposed between the radiation source and a proximal end of the handle. In some embodiments, the shield is constructed from a clear, transparent, or translucent material. In some embodiments, the material comprises plastic. In some embodiments, the beta radiation source is sealed. In some embodiments, the beta radiation source comprises a seed. In some embodiments, the beta radiation source provides a radiation dose of about 1000 cGy. In some embodiments, the beta radiation source provides a radiation dose between about 500 to 1500 cGy.

### BRIEF DESCRIPTION OF THE DRAWINGS

FIG. 1 is a schematic view of the apparatus of the present invention.

FIG. 2 is a perspective view of an example of an apparatus of the present invention (no shield is shown).

### DESCRIPTION OF PREFERRED EMBODIMENTS

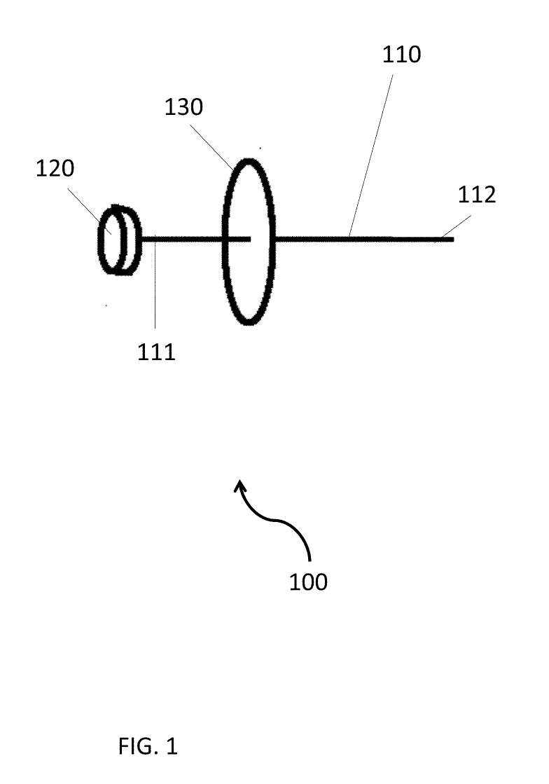

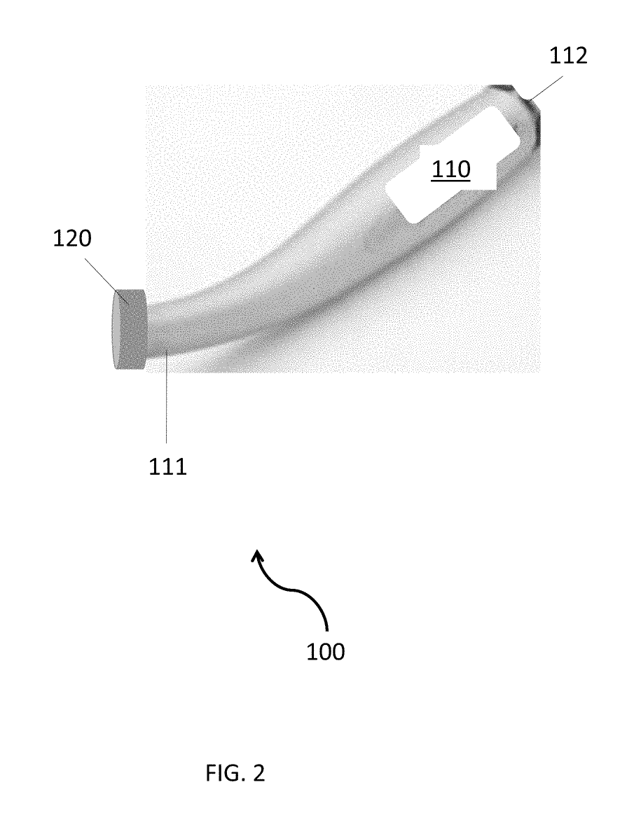

Referring now to FIG. 1-2, the present invention features methods and devices for the application of beta radiation following glaucoma surgery (e.g., Trabeculectomy). For example, the device, or applicator features a beta radiation source (e.g., Yttrium 90, Strontium 90, Potassium 32, or other radioisotope) adapted to emit beta radiation for effectively inhibiting wound reversion at the surgical fistula site following Trabeculectomy. Without wishing to limit the present invention to any theory or mechanism, it is believed that the beta radiation may inhibit fibroblast growth and/or other mechanisms of healing so as to maintain the patency of the surgical fistula.

As shown in FIG. 1-2, the applicator (100) comprises a handle (110), (e.g., a cannula, shaft, etc.) adapted to hold a radiation source. The handle (110) has a distal end (111) and a proximal end (112). In some embodiments, the radiation source is disposed at (or near) the distal end (111) of the handle (110), e.g., at the tip. In some embodiments, the radiation source is housed in the handle (110) and can optionally be advanced to the distal end (111) (or to near the distal end) as needed. Thus, a means for advancing the radiation source may be disposed in the handle (110).

The radiation source may be a beta radiation source (120) (e.g., Yttrium 90, Strontium 90, Potassium 32, or any other appropriate radiation source), however the radiation source is not limited to a beta radiation source (120). For example, the radiation source may comprise any appropriate radiation source and/or a combination of types of radiation sources.

In some embodiments, the radiation source (120) comprises strontium-90, yttrium-90, others, or a combination thereof. The present invention is not limited to the aforementioned radiation sources.

The radiation source may be constructed in a variety of ways. For example, in some embodiments, the radiation source, e.g., beta radiation source (120), is sealed. In some embodiments, the radiation source, e.g., beta radiation source (120), comprises a seed.

In some embodiments, the radiation source (e.g., radioisotope) is embedded in a matrix. The matrix may be sealed, e.g., with stainless steal, gold, or other appropriate containment. The seal or containment may allow the radioisotope (e.g., beta radiation) to exit the device, e.g., handle, and be deposited on the outer surface of the eye (e.g., sclera or overlying conjunctivae, etc.).

In some embodiments, the radiation source, e.g., beta radiation source (120), provides a radiation dose of about 1000 cGy. In some embodiments, the radiation source, e.g., beta radiation source (120), provides a radiation dose between about 500 to 1200 cGy. In some embodiments, the radiation source provides a radiation dose between about 250 to 1200 cGy. In some embodiments, the radiation source provides a radiation dose between about 250 to 1500 cGy. In some embodiments, the radiation source provides a radiation dose between about 500 to 1500 cGy. In some embodiments, the radiation source provides a radiation dose between about 750 to 1200 cGy. In some embodiments, the radiation source provides a radiation dose between about 750 to 1500 cGy. In some embodiments. the radiation source provides a radiation dose between about 250 to 2000 cGy. In some embodiments, the radiation source provides a radiation dose between about 500 to 2000 cGy. In some embodiments, the radiation source provides a radiation dose between about 1000 to 1200 cGy.

In some embodiments, the applicator (100) also comprises a shield (130) for shielding the physician's hand and other anatomy. The shield (130) may be disposed between the radiation source and a proximal end (112) of the handle (110) as shown in FIG. 1. In some embodiments, the shield (130) is constructed from a clear, transparent, or translucent material (e.g., plastic, acrylic, any appropriate material).

The present invention also features methods of preventing (or limiting, reducing) post-trabeculectomy wound reversion (e.g., healing of the surgical fistula). The method comprises exposing the trabeculectomy site to radiation, e.g., beta radiation, via an apparatus, e.g., via an apparatus (100) of the present invention. The radiation source, e.g., the beta radiation source (120), may function to prevent wound reversion.

In some embodiments, the method of claim comprises introducing a drug (e.g., mitomycin c, 5-fluorouracil (5FU), or other appropriate drug, e.g., anti-fibrotic drug, etc.) to the trabeculectomy wound. In some embodiments, the drug (e.g., mitomycin c, 5FU, other appropriate drug, etc.) is introduced after the wound is subjected to the beta radiation source (120). In some embodiments, drug (e.g., mitomycin c, 5FU, other appropriate drug, etc.) is introduced before the wound is subjected to the beta radiation source (120). In some embodiments, the drug (e.g., mitomycin c, 5FU, other appropriate drug, etc.) is introduced at about the same time the wound is subjected to the beta radiation source (120).

As used herein, the term “about” refers to plus or minus 10% of the referenced number. For example, an embodiment wherein the handle is about 5 inches in length includes a handle that is between 4.5 and 5.5 inches in length.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method of preventing post-trabeculectomy wound reversion, said method comprising:
(a) providing an applicator (100), the applicator (100) comprising a handle (110) with a distal end (111) and a beta radiation source (120) disposed on the distal end (111), the beta radiation source (120) comprises strontium-90, yttrium-90, potassium-32, or a combination thereof; and
(b) exposing a trabeculectomy wound to the beta radiation source (120), wherein the beta radiation source (120) functions to prevent wound reversion.

2. The method of claim 1, wherein the applicator (100) further comprises a shield (130) disposed between the radiation source (120) and a proximal end (112) of the handle (110).

3. The method of claim 2, wherein the shield (130) is constructed from a clear, transparent, or translucent material.

4. The method of claim 3, wherein the material comprises plastic.

5. The method of claim 1, wherein the beta radiation source (120) is sealed.

6. The method of claim 1, wherein the beta radiation source (120) comprises a seed.

7. The method of claim 1, wherein the beta radiation source (120) provides a radiation dose of about 1000 cGy.

8. The method of claim 1, wherein the beta radiation source (120) provides a radiation dose between about 500 to 1200 cGy.

9. The method of claim 1 further comprising the step of introducing a drug to the trabeculectomy wound after the wound is subjected to the beta radiation. source (120).

10. An applicator (100) for introducing radiation to a trabeculectomy wound, said applicator (100) comprising a handle (110) with a distal end (111) and a beta radiation source (120) disposed on the distal end (111), the beta radiation source (120) comprises strontium-90, yttrium-90, potassium-32, or a combination thereof.

11. The applicator (100) of claim 10, wherein the applicator further comprises a shield (130) disposed between the radiation source and a proximal end (112) of the handle (110).

12. The applicator (100) of claim 11, wherein the shield (130) is constructed from a clear, transparent, or translucent material.

13. The applicator (100) of claim 12, wherein the material comprises plastic.

14. The applicator (100) of claim 10, wherein the beta radiation source (120) is sealed.

15. The applicator (100) of claim 10, wherein the beta radiation source (120) comprises a seed.

16. The applicator (100) of claim 10, wherein the beta radiation source (120) provides a radiation dose of about 1000 cGy.

17. The applicator (100) of claim 10, wherein the beta radiation source (120) provides a radiation dose between about 500 to 1500 cGy.
